(19) United States
(12) Patent Application Publication
(10) Pub. No.: US 2018/0085410 A1
(43) Pub. Date: Mar. 29, 2018

(54) PROBIOTIC NUTRITIONAL SUPPLEMENT FOR ANIMALS

(71) Applicant: Tommy Stanley Watson, Palm Harbor, FL (US)

(72) Inventor: Tommy Stanley Watson, Palm Harbor, FL (US)

(21) Appl. No.: 15/277,596

(22) Filed: Sep. 27, 2016

(51) Int. Cl. A61K 35/741; A61K 35/742; A61K 35/747; A61K 35/745; A23K 10/18; A23K 20/163; A23K 50/40

(52) U.S. Cl. CPC A61K 35/741; A61K 35/742; A61K 35/747; A61K 2035/11; A23K 10/18; A23K 20/163; A23K 50/40; A61K 35/745

(57) ABSTRACT

A composition for promoting gastrointestinal and immune health in animals, such as canines and felines, contains a blend of dietary fiber, dietary sugar, and an effective amount of a probiotic microorganism including one or more unique strains of beneficial gastrointestinal microbial flora. At least 95% by weight of the composition can include certified organic ingredients.

### FIELD

The present technology relates to the field of probiotic nutritional supplementation for animals, and more particularly to an orally administered nutritional supplement including a dietary fiber, a dietary sugar, and a probiotic microorganism for canines or felines in powder form.

### INTRODUCTION

This section provides background information related to the present disclosure which is not necessarily prior art.

Probiotics, as defined by the World Health Organization (WHO), are live microorganisms which when administered in adequate amounts confer a health benefit on the host. These microorganisms have been studied for over 100 years for their effect on overall health and longevity of life. Isolation of lactic acid producing species, e.g. Lactobacillus spp. and Bifidobacterium spp., have led to dietary supplements and mostly dairy-based consumer products. Additionally, probiotics are defined as “micro-flora or microorganisms, which beneficially affect a host by improving its intestinal microbial balance” (Fuller, R; 1989; J. Applied Bacteriology, 66: 365-378).

In general, the term probiotic can be defined as “for life.” These beneficial microorganisms have earned this definition because they serve several beneficial functions in animals, including canines and felines. First, probiotics produce organic acids such as acetic acid, which can inhibit the growth of pathogenic bacteria such as E. coli, C. difficile, and H. pylori. Consequently, probiotic micro-flora can aid in the prevention and treatment of certain conditions, particularly digestive conditions, that can be caused by these pathogenic organisms. It is also believed that probiotic bacteria activate a host's own immune functions by mechanisms still not clearly understood. Considerable interest therefore exists in developing probiotic pet supplement products for animal nutrition.

There are three primary considerations when incorporating probiotic microorganisms into an animal nutritional supplement or food. First, the supplement must be in a physical form and component formulation such that it is palatable to the particular animal; e.g., feline or canine. Manufactures of human dietary supplements are not generally concerned with palatability of the supplement because human dietary supplements are generally swallowed whole. In other cases, humans can consume a product even though it does not taste good, as they experience or perceive they will experience a benefit for doing so. Animal probiotic supplements, conversely, must be palatable to the canine or feline consumer if compliance is to be achieved.

Second, animal owners are increasingly interested in the purity of the food or supplements consumed by their animals. An animal supplement should contain predominately active and beneficial ingredients and few if any artificial ingredients, including additives, fillers, and excipients. A problem arises in that pet supplements are usually found in capsule, powder, or chewable tablet form utilizing inexpensive binders, fillers, and flowing agents that provide no inherent benefit to the host animal. Such excipients and additives are typically selected because they are inexpensive and because their dryness and flowability are helpful in the manufacturing process. There is therefore a need to incorporate additives into the manufacture of a probiotic nutritional supplement that perform well in a particular manufacturing process (e.g., mixing and handling powder formulations), but at the same time provide one or more health benefits to the host animal.

Third, the probiotic microorganism must remain viable during the manufacturing process. Probiotics can be combined with an additive, filler, or bulking agent in order to add enough volume to the probiotic blend so that it can flow easily enough to be processed into a capsule, powder, or chewable tablet. Since probiotics can be particularly susceptible to moisture in the manufacturing process, common inexpensive fillers are normally used because they are known to be low in moisture. High moisture content in many powders can result in an unacceptable reduction in the survivability of the living probiotic microorganisms in the final formulation. This can be particularly true if the water content of the bulking powder is more than about 12% by weight.

There is accordingly a need for a probiotic nutritional supplement that can promote gastrointestinal and immune health in an animal, that is formed of a viable blend of one or more probiotic microorganisms, that is highly palatable to the animal, that is manufactured using additives that provide some inherent benefit to the animal, that has a low moisture content for stability and survivability of the probiotic microorganism blend, and that is free of artificial ingredients and nonorganic additives.

### SUMMARY

The present technology relates to probiotic nutritional supplements for animals, including compositions for promoting gastrointestinal and immune health in an animal and the manufacture of such compositions.

A composition for promoting gastrointestinal and immune health in an animal can include a dietary fiber, a dietary sugar, and a probiotic microorganism. The dietary fiber can include a water-soluble dietary fiber and/or a water-insoluble dietary fiber. The dietary fiber can include one or more types or sources of fiber, such as inulin, flax fiber, soy fiber, oat fiber, corn fiber, guar gum, gum Arabic, Larch bark, bean gum, gum acacia, pumpkin fiber, and/or chia fiber. The dietary sugar can include one or more monosaccharides, disaccharides, oligosaccharides, and polysaccharides. The dietary sugar can include certain oligosaccharides known to be energy sources for probiotic microorganisms, such as fructo-oligosaccharides, galacto-oligosaccharides, mannan-oligosaccharides, and/or isomalto-oligosaccharides. The probiotic microorganism can include one or more probiotic microorganisms selected from the genus Lactobacillus, the genus Bifidobacterium, the genus Enterococcus, the genus Saccharomyces, the genus Aspergillus, and/or the genus Bacillus. In certain embodiments, the probiotic microorganism includes a plurality of different probiotic microorganisms, where embodiments can include up to at least ten different probiotic microorganisms. The composition can have at least 1 billion colony forming units of the probiotic microorganism per serving of the composition. At least 95% by weight of the composition can be certified organic by the United States Department of Agriculture.

The composition can include living probiotic microorganisms that are unique strains known to be beneficial for gastrointestinal and immune health in various animals, including canines and felines. The dietary fiber can provide various advantages afforded by dietary fibers while avoiding various disadvantages associated with typical food additives or supplement fillers. The dietary sugar can promote the growth of the probiotic microorganisms of the composition following ingestion and/or can promote the growth of certain preexisting beneficial intestinal microorganisms by acting as an energy source for these microorganisms.

The present compositions and manufacture thereof can include formulations where at least about 95% by weight up to where substantially all (about 100% by weight) of the components in the composition are certified organic by the United States Department of Agriculture (USDA), including certification by the USDA Accredited Certifying Agents for the National Organic Program established in accordance with the Organic Foods Production Act of 1990 (7 U.S.C. § 6501 et seq.). Components used in making the present compositions can therefore exclude various artificial ingredients or additives. Moreover, the components can also be selected from dietary fiber, dietary sugar, and probiotic microorganisms approved for nutritional supplementation for animals according to the Association of Animal Feed Control Officials (AAFCO) of Champaign, Ill.

Further areas of applicability will become apparent from the description provided herein. The description and specific examples in this summary are intended for purposes of illustration only and are not intended to limit the scope of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. Except where otherwise expressly indicated, all numerical quantities in this description are to be understood as modified by the word “about” and all geometric and spatial descriptors are to be understood as modified by the word “substantially” in describing the broadest scope of the technology.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

The present technology is drawn to compositions for promoting gastrointestinal and immune health in an animal and methods of manufacturing such compositions. These include probiotic nutritional supplement compositions having at least one type of dietary fiber, at least one type of dietary sugar, and at least one type of probiotic microorganism. The compositions can be formulated as all-natural or substantially all-natural organic probiotic supplements for animals, including canines or felines. The compositions can be in a powder form and can include at least 95% by weight certified organic components, including a multiple probiotic blend of one or more unique strains of beneficial gastrointestinal micro-flora, and at least a powder ingredient or blend of powder ingredients selected from certified organic nutritional pet supplement ingredients as listed by AAFCO, or other naturally occurring certified organic components. One or more of the powder components can be present in an amount effective to act as a powder flowing agent while at the same time providing a dietary benefit to the host animal. The present technology can be distinguished over other animal supplements in that the present compositions can include an organic fiber, where at least 95% the formulation can include certified organic components, and the present compositions can include a blend of different viable probiotic microorganisms, including compositions having at one or more unique strains of beneficial gastrointestinal microorganisms selected from probiotic microorganisms accepted by AAFCO.

A composition for promoting gastrointestinal and immune health in an animal is provided that includes a dietary fiber, a dietary sugar, and a probiotic microorganism. The composition can include at least 95 percent by weight organic ingredients certified by the USDA. Remaining components can include non-certified organic substances allowed as ingredients in or on products labeled as “organic,” as specified by 7 U.S.C. § 205.605. As used herein, the term “certified organic” identifies components that are certified “organic” by the USDA and the accredited certifying agents for the National Organic Program, established in accordance with the Organic Foods Production Act of 1990 (7 U.S.C. § 6501 et seq.). Certified organic ingredients are also compliant with processing standards for growing, storing, processing, packaging, and shipping to avoid synthetic chemicals, genetically modified organisms, irradiated materials and sewage sludge.

In some embodiments, the composition can include up to 100 percent by weight of certified organic ingredients in the final formulation. Organic compliant, or non-organic but acceptable substances (as listed under 7 U.S.C. § 205.605), can be used in the formulation of the composition. If needed, small amounts of organic compliant ingredients or flowing agents or processing aids, etc. can be used to enhance the mixing, flowing, general manufacturing, or processing of the composition. In general, the effective amounts of mixing, manufacturing, or flow aids (if used at all) are about 5% by weight or less of the composition, such that the final composition can be labeled “organic,” as per USDA certification requirement. These additional processing and manufacturing aids can be added in any order or manner necessary to aid in the preparation of the present compositions.

Generally, certified organic components or products are those that do not contain any artificial flavoring, coloring or chemical preservative (as defined by 21 C.F.R. § 01.22) or any other artificial or synthetic ingredient. Additionally, certified organic components or products generally are not subjected to processing or are only subjected to minimal processing. Excluded from the category of certified organic components are most of the commonly employed powder excipients including non-nutritive derivatives and other synthetic or highly processed ingredients.

A number of components, including select certified organic components, can provide an energy source or food for viable probiotic microorganisms. Among these components that can serve as an energy source are those categorized as oligosaccharides and some dietary fibers. The present technology can be formulated to include the addition of an energy source in the form of oligosaccharides and/or dietary fiber as long as two criteria are met: 1) the addition of the oligosaccharides or dietary fiber does not cause the final formulation to fall below 95% by weight of certified organic components; and 2) the oligosaccharides and/or dietary fiber are considered to be nutrient suitable for an animal diet (e.g., canine or feline diet) according to AAFCO. The present compositions can therefore include compositions where at least 95% by weight of the composition is certified organic by the USDA. In various embodiments, the compositions can therefore include where the dietary fiber and the dietary sugar comprise at least 95% by weight of the composition. The dietary fiber and the dietary sugar can be certified organic by the United States Department of Agriculture and can be approved for animal use by AAFCO.

The composition for promoting gastrointestinal and immune health in an animal can include one or more types of dietary fiber. The term “dietary fiber” as used herein refers to the part of a plant that is indigestible to the animal. There are two types of fiber: water-soluble (can be dissolved in water) and water-insoluble (does not dissolve in water). Fiber used in the present technology can include one or more types of soluble fiber, insoluble fiber, or both soluble fiber and insoluble fiber. Specific examples of suitable dietary fiber useful in the present technology include one or more of inulin (from chicory or agave), flax fiber, soy fiber, oat fiber, corn fiber, guar gum, gum Arabic, Larch bark, bean gum, and gum acacia. Certain embodiments include where the dietary fiber is a member selected from the group consisting of: inulin, flax fiber, soy fiber, oat fiber, corn fiber, guar gum, gum Arabic, Larch bark, bean gum, gum acacia, pumpkin fiber, chia fiber, and combinations thereof. Other embodiments include where the dietary fiber includes at least two members selected from the group comprising: inulin, flax fiber, soy fiber, oat fiber, corn fiber, guar gum, gum Arabic, Larch bark, bean gum, gum acacia, pumpkin fiber, and chia fiber.

In addition to their use in manufacturing of the present compositions, dietary fibers can provide one or more health benefits to the host animal. Soluble fiber can be fermented in the intestine of the animal and can function as a prebiotic, where a “prebiotic” is a substance that induces the growth or activity of microorganisms in the gastrointestinal tract of the animal. Insoluble fiber can be metabolically inert and can provide bulk as it moves through the intestine of the animal to ease defecation. Dietary fiber used herein can include multiple types of soluble fiber and/or insoluble fiber. Thus, the dietary fiber used in the present technology can aid in the manufacture of the present compositions and can also provide digestive benefits to the host animal.

The composition for promoting gastrointestinal and immune health in an animal can include one or more types of dietary sugar. The term “dietary sugar” as used herein refers to carbohydrates, including various saccharides in monomeric and polymeric forms, such as monosaccharides, disaccharides, oligosaccharides and polysaccharides. Examples of monosaccharides include fructose, glucose, and galactose. Examples of disaccharides include sucrose, lactose, and maltose. The term “oligosaccharide” as used herein refers to a saccharide polymer having three to ten monosaccharide units linked together. Examples of suitable oligosaccharides include fructo-oligosaccharides (FOS), galacto-oligosaccharides (GOS), mannan-oligosaccharides (MOS), and isomalto-oligosaccharides (MOS). The oligosaccharide can be only partially digestible by the animal. The undigested part of the oligosaccharide can serve as an energy source to help support intestinal microflora (i.e. microorganisms living in the digestive tract, many of which perform useful functions). Oligosaccharides can also increase the quantity of beneficial microorganisms in the colon of the animal while also reducing the quantity of harmful microorganisms. In certain cases, the oligosaccharide can facilitate growth and/or colonization of the probiotic microorganism from the present composition within the host animal. The dietary sugar can include more than one type of carbohydrate, including various saccharides in monomeric and/or polymeric forms.

The composition for promoting gastrointestinal and immune health in an animal can include one or more probiotic microorganisms. There are thousands of probiotic strains commercially available worldwide. The selection of a probiotic microorganism for use in the present compositions is dependent on several factors. First, the probiotics microorganism (as identified by genus and species) can be selected from the list of approved probiotic microorganism strains found in the official publication of the Association of Animal Feed Control Officials (AAFCO). In order to make commercially available a probiotic pet supplement in the United States, the probiotic strains used in the present technology can be selected from the AAFCO list, which currently includes the following members: Lactobacillus acidophilus, Lactobacillus bulgaricus, Lactobacillus casei, Lactobacillus plantarum, Lactobacillus helveticus, Lactobacillus reuteri, Lactobacillus delbruekii, Lactobacillus fermentum, Lactobacillus lactis, Lactobacillus brevis, Bifidobacterium bifidum, Bifidobacterium animalis, Bifidobacterium longum, Bifidobacterium infantis, Bifidobacterium thermophilum, Enterococcus faecium, Enterococcus lactis, Saccharomyces cerevisiae, Aspergillus niger, Aspergillus oryzae, Bacillus coagulans, Bacillus licheniformis, Bacillus pumilus, and Bacillus subtilis. It should be noted that the AAFCO list of probiotic microorganisms is annually reviewed and can be modified. As such, the selection of strains of probiotic microorganisms for the present technology is not to be limited in any way by the currently approved probiotic microorganisms by AAFCO. The only limitation is that the strains selected be approved for consumption by animals (e.g., canines and felines) according to the Association of Animal Feed Control Officials (AAFCO) or by the prevailing state or federal regulations or guidelines in place at the time.

Second, the probiotic microorganisms can be selected such that they deliver an effective level of potency per serving in a given composition. Potency of probiotic microorganisms per gram and serving sizes of probiotic microorganisms in some supplements can vary significantly. The present compositions can include probiotic microorganisms ranging from 100 million colony forming units per serving to 100 billion colony forming units per serving. The delivered potency per serving can be some multiple or factor of 1 billion colony forming units per serving. Potency per serving for canines and felines can range from at least 2 billion colony forming units per serving up to 100 billion colony forming units per serving. Potency of 5 billion colony forming units per serving up to 50 billion colony forming units per serving being the most generally accepted clinical delivery range. This is not intended to limit the potency per serving of the present invention only to these ranges. Clinically accepted levels of potency per serving can change over time as new clinical data becomes available. In the present technology, there can be a clinically effective level of one or more viable probiotic microorganisms available per serving to have a positive impact on the host animal. Various embodiments of the composition can include at least 1 billion colony forming units of the probiotic microorganism per serving of the composition, at least 10 billion colony forming units of the probiotic microorganism per serving of the composition, and at least 100 billion colony forming units of the probiotic microorganism per serving of the composition.

Third, the probiotic microorganism can be selected according to a potency per gram of material. Potency is generally measured as number of cultures per gram or number of colony forming units per gram. For example, a probiotic microorganism can be formulated such that it contains 1 billion cultures in each gram of final product. Additionally, the probiotic microorganism can be formulated at 100 billion cultures in each gram of final product. Therefore, the latter formulation would be considered to be 100 times more potent than the former. Different probiotic microorganism strains can vary greatly in potency or number of cultures per gram. In order to have a composition according to the present technology obtain an “organic” certification under the USDA, the amount of probiotic microorganisms should be less than 5% by weight of the final composition, leaving 95% by weight or more of the composition formed of organic ingredients, including the dietary fiber and dietary sugar, for example. Therefore, only probiotic microorganism strains of sufficient potency can be utilized in the present formulations. Less potent probiotic microorganism strains can be too voluminous to deliver an effectively potent level of probiotics and may not allow for at least 95% by weight of the final formulation to be organic components to achieve organic certification of the formulation.

In certain embodiments, the probiotic microorganism can include a member selected from the group consisting of: the genus Lactobacillus, the genus Bifidobacterium, the genus Enterococcus, the genus Saccharomyces, the genus Aspergillus, the genus Bacillus, and combinations thereof. The probiotic microorganism can also include a member selected from the group consisting of: Lactobacillus acidophilus, Lactobacillus bulgaricus, Lactobacillus casei, Lactobacillus plantarum, Lactobacillus helveticus, Lactobacillus reuteri, Lactobacillus delbruekii, Lactobacillus fermentum, Lactobacillus lactis, Lactobacillus brevis, Bifidobacterium bifidum, Bifidobacterium animalis, Bifidobacterium longum, Bifidobacterium infantis, Bifidobacterium thermophilum, Enterococcus faecium, Enterococcus lactis, Saccharomyces cerevisiae, Aspergillus niger, Aspergillus oryzae, Bacillus coagulans, Bacillus licheniformis, Bacillus pumilus, Bacillus subtilis, and combinations thereof. The probiotic microorganism can also include from 1 to 24 of the members of the preceding group, where some embodiments include at least two members of the preceding group, other embodiments include at least ten members of the preceding group, and further embodiments include all 24 members of the preceding group.

Compositions for promoting gastrointestinal and immune health in an animal according to the present technology can be formulated in various ways and in various forms. Examples of serving sizes can include amounts ranging from about 0.1 grams to about 100 grams. In certain embodiments, the serving size can be about 4 grams. The respective potency of the probiotic microorganism and the amounts of dietary fiber and dietary sugar can be tailored based on the desired percentages by weight for each component. The water content of the composition can be less than about 12% by weight. Probiotic microorganisms can be sensitive to moisture in the manufacturing process, where high moisture content can reduce the survivability and viability of living probiotic microorganisms during manufacture and in the final composition. The composition can therefore be formed using dietary fiber, dietary sugar, and/or probiotic microorganisms of sufficiently low moisture content so that the final composition has less than about 12% by weight water. In certain embodiments, the composition can have less than about 0.7% by weight water, where further embodiments include less than 0.5% by weight water.

The composition can be formed as a powder where components of the composition are admixed or combined in various ways. Individual particles of the powder can include an agglomerate of multiple components of the composition, individual particles of the powder can include individual components of the composition, or particles of the powder can include a mixture thereof. For example, the components can be mixed, granulated or extruded, pulverized, and screened or sorted to provide a relatively uniform powder having particles that include agglomerations of the composition components. Alternatively, the components can be in powder form and mixed to form a powder of combined components, where each particle is substantially formed an individual component. The handling and processing of various components of the composition can present flow issues. The dietary fiber and/or the dietary sugar can function as a flowing agent to ensure the powder composition can be processed as well as metered or dispensed in a consistent fashion.

Formulations of the compositions for promoting gastrointestinal and immune health in an animal can include various amounts of the dietary fiber, the dietary sugar, and the probiotic microorganism. The dietary fiber can comprise from about 80% by weight to about 95% by weight of the composition. The dietary sugar can comprise about 0.5% by weight to about 2% by weight of the composition. The probiotic microorganism can comprise about 0.5% by weight to about 5% by weight of the composition. Certain formulations include where the dietary fiber comprises from about 80% by weight to about 95% by weight of the composition, the dietary sugar comprises about 0.5% by weight to about 2% by weight of the composition, the probiotic microorganism comprises about 0.5% by weight to about 5% by weight of the composition, the composition includes at least 1 billion colony forming units of the probiotic microorganism per serving, and/or the composition includes less than about 12% by weight of water.

Additional ways to make the present compositions include adding the various component sequentially or in a single batch and blending or mixing the components to form a substantially homogenous composition. When made in powder form, the composition can be packaged in bulk or can be packaged into individual servings. Packaging can include various moisture-barrier containers, including various plastic containers or foil packets. A desiccant can be included in the packaging in order to maintain less than about 12% by weight of water and preserve the viability of the probiotic microorganism. The present compositions can be used by dispensing a serving onto food for the animal. For example, a powdered composition can be admixed with canine or feline food kibble or pellets or can be sprinkled on top of the animal's food. In certain cases, the present compositions can be included in the preparation of animal food. Other embodiments include adding a serving of the present composition to a serving of animal food. In this manner, the present composition can be sealed in a moisture-barrier container to maintain less than about 12% by weight water while the animal food may be packaged differently and include more than 12% by weight of water.

In accordance with certain aspects of the present technology, processes for making the present compositions are provided where the resulting composition contains at least 95% certified organic ingredients by weight. The process can include the step of selecting unique and highly viable probiotic strains that are capable of delivering an effective enough number of strains per given gram of the composition such that the final composition delivers enough probiotic cultures to have a positive impact on the host animal while at the same time taking up 4.99% or less of the final formulation by weight. At least 95% of the composition by weight is added using certified organic components made of either individually or a combination of certified organic dietary fibers and/or certified organic or non-organic dietary sugars in the form of one or more monosaccharides, disaccharides, oligosaccharides, or polysaccharides. All of the components of the composition can be combined in a manner that does not negatively impact the viability of the probiotic microorganism. The process can be performed such that the probiotic potency of the probiotic microorganism blend remains stable enough to survive the manufacturing process and still provide a benefit to the host animal. The process can therefore result in composition where the final formulation can be certified by the designation “organic” by one of the USDA Accredited Certifying Agents for the National Organic Program established in accordance with the Organic Foods Production Act of 1990 (7 U.S.C. § 6501 et seq.).

The examples of dietary fibers and dietary sugars included herein can be provided as organic ingredients in powder form and have been found to possess good manufacturing characteristics specifically in the formulation of probiotic nutritional supplements for animals that can promote gastrointestinal and immune health. As described, a concern in manufacturing probiotic supplements is the moisture content of the components, including any bulking agents, used in the manufacturing process. It has been discovered that the powdered dietary fibers described herein can have the desired low moisture characteristics needed in the manufacturing of pet probiotic nutritional supplements. Furthermore, these powders are known to mix well, have good particle dynamics, and limited dusting characteristics.

Several benefits and advantages are afforded by the present technology over other animal supplements, including probiotic supplements designed for administration to animals. Probiotics are increasingly administered by animal and pet owners for a variety of reasons. There is a growing body of data in the veterinary literature suggesting that probiotic microorganism strains and combinations of strains demonstrate an ability of probiotics to alter the fecal microbiome and produce a positive host response. Diarrheal diseases are often characterized by microbiomic changes (Bell et al 2009). Recent investigations in dogs suggest that probiotic combinations can have a positive impact on the microbiome of canines and felines. Furthermore, studies have identified possible immunoregulatory effects of probiotic strains (Benyacoub et al. 2003). Single-strain probiotics have reduced feline diarrheal episodes in animal shelters (Bybee et al 2011), shortened time to resolution of canine idiopathic diarrhea by 40% (Kelley et al. 2009), and induced changes in clostridial numbers (Biagi et al 2007). In recent literature, a combination probiotic product shortened signs of canine gastroenteritis as compared to placebo (1.3d vs. 2.2d, Herstad et al. 2010). The literature therefore indicates that the safety and efficacy of commercial probiotic products are likely unique to a specific bacterium or combination of bacteria delivered as an intended probiotic. The present technology can include the use of a blend of different viable probiotic microorganisms that realize multiple benefits to the animal, including synergistic and supra-additive effects that include treating a particular gastrointestinal and/or immune issue in the animal while simultaneously maintaining a balance of intestinal microorganisms, thereby reducing side effects of perturbing the endogenous intestinal microorganism community in the animal. For example, in certain instances, administration of a single strain of probiotic microorganism may result in an overrepresentation of that strain, which may be advantageous for one gastrointestinal and/or immune issue, but not for another gastrointestinal and/or immune issue. The present technology can overcome these issues and even address unforeseeable gastrointestinal and/or immune issues by providing multiple strains of probiotic microorganisms at high levels of colony forming units, enabling a surprising level of potency and efficacy in mitigating gastrointestinal and immune issues in the animal.

A variety of animal supplements in powder, tablet, and capsule form are available. However, animal or pet “probiotic” supplements are relatively new and found in relatively limited number. Certain probiotic pet supplements currently available include ingredients, binders, fillers, and/or other flowing agents and excipients that either are not or cannot be certified as organic ingredients pursuant to the requirements of the Organic Foods Production Act of 1990 (7 U.S.C. § 6501 et seq.). Furthermore, certain pet probiotic supplements use as their base a carbohydrate in the form of the food additive maltodextrin. Maltodextrin is used in probiotic supplementation formulations because it is very inexpensive and relatively low in moisture content. Maltodextrin is thought to be a convenient carrier for probiotics, which require low moisture to remain viable. However, maltodextrin does not impart a significant health benefit to the host animal. Conversely, the present compositions provide a dietary fiber and/or a dietary sugar for use in formulating a viable probiotic blend, where each can provide a health benefit to the host animal and is also available in certified organic form.

Other attempts to satisfy the demand for animal probiotic supplement products have generally involved preparation of hard 2-piece shell capsules, comprised of animal gelatin or plant-based material. However, these capsules are not certified organic since the raw materials used to make the capsules are not organic or organic compliant, therefore the total capsule does not meet the at least 95% certified organic requirement by the USDA. Moreover, in terms of consumer preference as it relates to animal supplement dosage forms, hard shell gelatin capsules are generally considered less desirable than powders because of the difficulty of getting compliance from the animal. In contrast, the present technology can provide all-natural or substantially all-natural pet probiotic supplements in a powder form comprised entirely or almost entirely of certified organic ingredients. Specifically, the present technology can provide powder formulations having a suitable dryness and processing characteristics, such as flowability, that afford proper powder mixing of components and maintain viability of the probiotic microorganisms.

### EXAMPLES

Composition formulations according to the present technology include the following examples.

Formulation A: probiotic nutritional supplement powder for promoting gastrointestinal and immune health in an animal, based on a 3.7 gram per serving size having:
3.515 g certified organic dietary fiber,
0.035 g fructo-oligosaccharides (FOS), and
0.150 g viable probiotic microorganism including at least 1 billion colony forming units.

Formulation B: probiotic nutritional supplement powder for promoting gastrointestinal and immune health in an animal, based on a 3.7 gram per serving size having:
3.00 g certified organic soluble dietary fiber,
0.52 g certified organic insoluble dietary fiber,
0.03 g fructo-oligosaccharides (FOS), and
0.15 g viable probiotic micro-flora blend including ten strains.

Formulation C: probiotic nutritional supplement powder for promoting gastrointestinal and immune health in an animal, based on a 3.7 gram per serving size having:
3.200 g certified organic soluble dietary fiber blend,
0.315 g insoluble dietary fiber, and
0.185 g viable probiotic microorganism blend.

Formulation D: probiotic nutritional supplement powder for promoting gastrointestinal and immune health in an animal, based on a 3.7 gram per serving size having:
3.515 g certified organic dietary fiber,
0.035 g galacto-oligosaccharides (GOS), and
0.150 g viable probiotic microorganism blend of at least 10 strains.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

1. A composition for promoting gastrointestinal and immune health in an animal, the composition comprising:
a member selected from the group consisting of a dietary fiber, a dietary sugar, and combinations thereof; and
a probiotic microorganism, wherein the probiotic microorganism comprises about 0.1% by weight to about 5% by weight of the composition.

2. The composition of claim 1, wherein at least 95% by weight of the composition is certified organic by the United States Department of Agriculture.

3. A composition for promoting gastrointestinal and immune health in an animal, the composition comprising:
a member selected from the group consisting of a dietary fiber, a dietary sugar, and combinations thereof, wherein the member comprises at least 95% by weight of the composition; and
a probiotic microorganism.

4. The composition of claim 1, wherein the dietary fiber and the dietary sugar are certified organic by the United States Department of Agriculture.

5. The composition of claim 1, wherein the dietary fiber is a member selected from the group consisting of: inulin, flax fiber, soy fiber, oat fiber, corn fiber, guar gum, gum Arabic, Larch bark, bean gum, gum acacia, pumpkin fiber, chia fiber, and combinations thereof.

6. The composition of claim 1, wherein the dietary fiber includes at least two members selected from the group comprising: inulin, flax fiber, soy fiber, oat fiber, corn fiber, guar gum, gum Arabic, Larch bark, bean gum, gum acacia, pumpkin fiber, and chia fiber.

7. The composition of claim 1, wherein the dietary fiber includes a water-soluble dietary fiber and a water-insoluble dietary fiber.

8. The composition of claim 1, wherein the dietary sugar includes an oligosaccharide.

9. The composition of claim 1, wherein the dietary sugar is a member selected from the group consisting of: fructo-oligosaccharides, galacto-oligosaccharides, mannan-oligosaccharides, isomalto-oligosaccharides, and combinations thereof.

10. The composition of claim 1, wherein the probiotic microorganism is a member selected from the group consisting of: the genus Lactobacillus, the genus Bifidobacterium, the genus Enterococcus, the genus Saccharomyces, the genus Aspergillus, the genus Bacillus, and combinations thereof.

11. The composition of claim 1, wherein the probiotic microorganism is a member selected from the group consisting of: Lactobacillus acidophilus, Lactobacillus bulgaricus, Lactobacillus casei, Lactobacillus plantarum, Lactobacillus helveticus, Lactobacillus reuteri, Lactobacillus delbruekii, Lactobacillus fermentum, Lactobacillus lactis, Lactobacillus brevis, Bifidobacterium bifidum, Bifidobacterium animalis, Bifidobacterium longum, Bifidobacterium infantis, Bifidobacterium thermophilum, Enterococcus faecium, Enterococcus lactis, Saccharomyces cerevisiae, Aspergillus niger, Aspergillus oryzae, Bacillus coagulans, Bacillus licheniformis, Bacillus pumilus, Bacillus subtilis, and combinations thereof.

12. The composition of claim 1, wherein the probiotic microorganism includes at least two members selected from the group consisting of: Lactobacillus acidophilus, Lactobacillus bulgaricus, Lactobacillus casei, Lactobacillus plantarum, Lactobacillus helveticus, Lactobacillus reuteri, Lactobacillus delbruekii, Lactobacillus fermentum, Lactobacillus lactis, Lactobacillus brevis, Bifidobacterium bifidum, Bifidobacterium animalis, Bifidobacterium longum, Bifidobacterium infantis, Bifidobacterium thermophilum, Enterococcus faecium, Enterococcus lactis, Saccharomyces cerevisiae, Aspergillus niger, Aspergillus oryzae, Bacillus coagulans, Bacillus licheniformis, Bacillus pumilus, Bacillus subtilis.

13. The composition of claim 1, wherein the probiotic microorganism includes at least ten members selected from the group consisting of: Lactobacillus acidophilus, Lactobacillus bulgaricus, Lactobacillus casei, Lactobacillus plantarum, Lactobacillus helveticus, Lactobacillus reuteri, Lactobacillus delbruekii, Lactobacillus fermentum, Lactobacillus lactis, Lactobacillus brevis, Bifidobacterium bifidum, Bifidobacterium animalis, Bifidobacterium longum, Bifidobacterium infantis, Bifidobacterium thermophilum, Enterococcus faecium, Enterococcus lactis, Saccharomyces cerevisiae, Aspergillus niger, Aspergillus oryzae, Bacillus coagulans, Bacillus licheniformis, Bacillus pumilus, Bacillus subtilis.

14. (canceled)

15. The composition of claim 1, wherein the composition includes at least 1 billion colony forming units of the probiotic microorganism per serving of the composition.

16. The composition of claim 1, wherein the composition includes at least 10 billion colony forming units of the probiotic microorganism per serving of the composition.

17. The composition of claim 1, wherein the composition includes at least 100 billion colony forming units of the probiotic microorganism per serving of the composition.

18. The composition of claim 1, wherein the composition is a powder.

19. The composition of claim 18, wherein the dietary fiber or dietary sugar functions as a flowing agent.

20. The composition of claim 1, wherein the composition includes less than about 12% by weight of water.

21. The composition of claim 1, wherein the dietary fiber comprises from about 80% by weight to about 95% by weight of the composition.

22. The composition of claim 1, wherein the dietary sugar comprises about 0.5% by weight to about 2% by weight of the composition.

23. (canceled)

24. The composition of claim 1, wherein the dietary fiber comprises from about 80% by weight to about 95% by weight of the composition, the dietary sugar comprises about 0.5% by weight to about 2% by weight of the composition, the probiotic microorganism comprises about 0.5% by weight to about 5% by weight of the composition, the composition includes at least 1 billion colony forming units of the probiotic microorganism per serving of the composition, and the composition includes less than about 12% by weight of water.

25. A composition for promoting gastrointestinal and immune health in an animal, the composition comprising:
a dietary fiber, wherein the dietary fiber comprises from about 80% by weight to about 95% by weight of the composition; and
a probiotic microorganism
